(19) United States
(12) Patent Application Publication
(10) Pub. No.: US 2008/0255582 A1
(43) Pub. Date: Oct. 16, 2008

(54) Methods and Template Assembly for Implanting an Electrode Array in a Patient

(76) Inventor: John F. Harris, Bellevue, WA (US)

Correspondence Address: NEUROVISTA / SHAY GLENN, 2755 CAMPUS DRIVE, SUITE 210, SAN MATEO, CA, 94403, US

(21) Appl. No.: 11/734,190

(22) Filed: Apr. 11, 2007

(51) Int. Cl. A61B 19/00

(52) U.S. Cl. 606/129

(57) ABSTRACT

Templates and their methods of use for creating a plurality of holes or openings at predetermined locations in the skull of a patient, wherein the plurality of holes or openings are configured to receive a plurality of electrodes that may monitor brain activity signals from the patient and/or deliver a neuromodulation therapy to the patient.

### BACKGROUND OF THE INVENTION

Electrodes can be used to electrically monitor and/or stimulate a patient by positioning them on or within the patient's head. The electrodes may be used to monitor brain signals such as EEG or MEG, and/or may be used to delivery neuromodulation therapy to portions of the brain. The electrodes may be positioned on or within the scalp, or above, within, or under the skull, including on or within portions of the brain.

Conventional methods of positioning electrodes in a patient's skull comprise creating a burr hole in the skull, positioning the electrodes above or underneath the dura mater, and advancing the electrode array, typically a strip electrode, until the physician believes that the electrodes are placed in an appropriate position. More invasive methods of implanting electrodes require a craniotomy, in which a portion of the patient's skull is removed, and the electrode array, typically grid electrodes, are placed above or underneath the dura mater.

A number of different methods have been described in the patent literature. For example, U.S. Patent Application Publication No. 2005/0070810 to Kennedy is related to detecting neural activity using conductive skull screws implanted in the skull of a patient. The locations of the skull screws are chosen so that a change in neural electrical potential between the first and second conductive skull screws occurs when a patient performs a neural task.

While not related to implanting electrodes, U.S. Patent Application Publication No. 2006/0291968 to Greenberg discloses a template that may be used in stereotaxic surgery.

The deficiency of these systems and with conventional methods of electrode placement in a patient's skull is that they do not address the need for accurately placing a plurality of electrodes that are in a preformed pattern or configuration in the patient's skull.

### SUMMARY OF THE INVENTION

The present invention provides methods and systems for identifying relative positions of burr holes in the patient's skull. The positions of the burr holes identified by a template will typically correspond to the orientation of at least some of the individual electrode contacts from a selected electrode array. The selected electrode array may be selected from a bank of preformed templates or the selected electrode array may be custom made for the particular patient. Since most of the electrode arrays will typically have between 4 electrodes and 16 electrodes, it is desirable to have the burr holes be created in a position that corresponds to the electrode contacts of the electrode array so that the electrode contacts may be positioned in and/or through the burr holes created in the patient's skull.

One aspect of the invention is a template for creating a plurality of openings at locations in the skull of a patient, wherein the plurality of openings are configured to receive an electrode array. The template comprises a substrate configured to be coupled to an outer surface of the patient's skull. The template also includes a plurality of markers in a pattern on the substrate, wherein the markers are configured to identify locations for creating a plurality of openings in the skull, and the openings are configured to receive an electrode array comprising a plurality of electrodes that are positioned in a pattern that corresponds to the pattern of the plurality of markers.

The substrate can be configured to be removably or permanently coupled to the outer surface of the skull. In some embodiments the plurality of markers are configured to identify patient-specific locations. In some embodiments the plurality of markers includes a plurality of channels in the substrate, wherein the plurality of channels are adapted to receive a drill element, such as a drill bit. The plurality of markers may also comprise a plurality of visual markings on the substrate.

In some embodiments the locations for creating a plurality of openings are based on the location of the patient's seizure foci. The plurality of electrodes can be configured to monitor brain signals and/or deliver a neuromodulation therapy (e.g., electrical stimulation, drug delivery, etc.) to the patient's brain.

Another aspect of the invention is a system for implanting a plurality of electrodes at locations within a patient's skull. The system comprises a template comprising a substrate and a plurality of markers in a pattern, wherein the substrate is configured to be coupled to the outer surface of the skull, and wherein the markers are configured to identify locations at which to create a plurality of openings in the skull. The system also includes an electrode array comprising a plurality of electrodes that are in a pattern that corresponds to the pattern of the plurality of markers. The plurality of openings in the skull is configured to receive the plurality of electrodes.

The substrate can be configured to be removable or permanently coupled to the outer surface of the skull. In some embodiments the plurality of markers comprises a plurality of channels in the substrate, wherein the plurality of channels are configured to receive a drill element such as a drill bit. The plurality of markers may also comprise a plurality of visual markings on the substrate. In some embodiments the plurality of markers are configured to identify patient-specific locations. In some embodiments the locations are based on the location of seizure foci.

The system may also include a template lid shaped to mate with the template, wherein the electrode array is configured to be disposed between the template lid and the template when the template lid mates with the template.

The plurality of electrodes can be configured to monitor brain signals and/or to deliver a neuromodulation output to the patient's brain. The electrode array can include at least one lead and the template can include at least one lead channel adapted to mate with the at least one lead.

In some embodiments, the system may further include an implantable medical device configured to be implanted below the head of the patient. The electrode array may include at least one lead that is connected to the implanted medical device, or the electrodes may be wirelessly connected to the implanted device.

Another aspect of the invention is a method for implanting a plurality of electrodes at locations in a patient's skull. The method includes positioning a template comprising a plurality of markers on an exterior surface of the skull, the markers identifying a plurality of locations on the skull for creating a plurality of openings. The method also includes creating the plurality of holes in the skull at the plurality of locations, and positioning a plurality of electrodes in the plurality of holes.

In some embodiments the method also includes selecting a template from a plurality of templates based on the determination of the plurality of locations. In other embodiments, the template may be custom-made for the particular patient.

In some embodiments the markers comprise a plurality of channels in the template, and creating a plurality of openings comprises drilling into the skull through the plurality of channels. In other embodiments the markers comprises a plurality of channels and the method also includes creating a visual reference on the exterior surface of the skull at the identified locations after positioning the template on the exterior surface of the skull, and removing the template from the exterior surface of the skull before creating a plurality of openings.

Typically, the holes or openings are created above or over seizure foci. After the holes or openings are created in the skull, a plurality of electrodes may be positioned in the plurality of holes or opening. After the electrodes are positioned in the holes or openings, a lid may be placed on the template, thereby securing the electrode array. The method may also include connecting the plurality of electrodes to an implantable medical device.

For a further understanding of the nature and advantages of the present invention, reference should be made to the following description taken in conjunction with the accompanying drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary template with a plurality of markers.

FIG. 2 shows an exemplary template with an opening or channel for creating an opening in the skull of a patient.

FIG. 3 illustrates an exemplary system of the present invention including a template for creating a plurality of openings in the skull an electrode array.

FIG. 4 shows exemplary electrode arrays that may be positioned in openings created in the skull of a patient.

FIG. 5 shows an exemplary system that may be used with the template of the present invention.

FIG. 6 illustrates an example of a kit embodied by the present invention.

FIG. 7 illustrates one method encompassed by the present invention.

FIG. 8 illustrates another method encompassed by the present invention.

### DETAILED DESCRIPTION OF THE INVENTION

The present invention is related to methods and devices for creating openings in a patient's skull for implanting an array of electrodes. The pattern of the openings in the patient's skull is preferably predetermined using a template. The template will generally be designed for a specific electrode array, but may also be customized for an individual patient. The template may be coupled, either temporarily or permanently, to the skull and will provide, with markings, an indication as to where to create the openings in the skull. After the openings are created, the template may be left in place or removed, and the electrodes can then be positioned within the openings. One goal of the present invention is to accurately place a plurality of electrodes in a patient's skull such that the electrodes are positioned in predetermined locations.

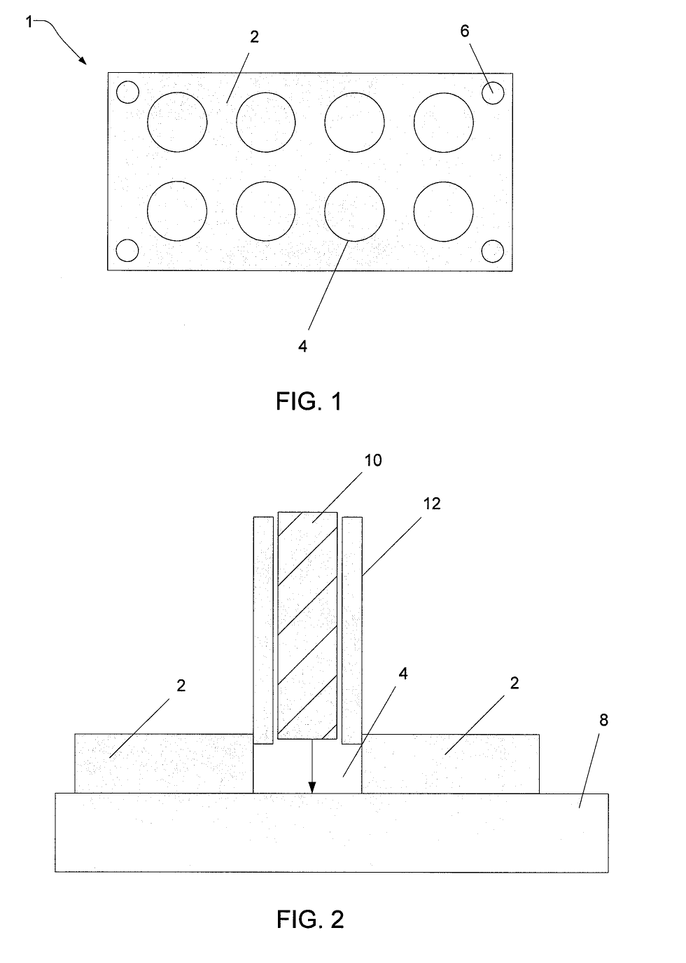

FIG. 1 shows an exemplary template of the present invention. Template 1 comprises a substrate 2 and a plurality of markers 4 in a pattern. The markers 4 may be, for example, visual markings, openings/channels, or other means for identifying locations where openings should be created in the skull of the patient. In embodiments in which the markers 4 comprise openings, the openings will generally be the same size or larger than a drill bit that can be used to create the opening and/or the electrode that is to be implanted in the skull. As shown in FIG. 2, marker 4, shown as an opening, may be large enough to accommodate a protective sheath 12 that is placed around drill bit 10 which can be used to drill holes in the skull 8. Protective sheath 12 may be used to guide drill bit 10 and/or prevent any undesirable abrasions to the template 1 during the drilling procedure.

As used herein, the term “skull” includes the bones within a patient's head, including the cranium, facial bones, jaw bones, and crossbones.

The markers 4 may have a generic pattern that has conventional or predetermined spacing between the markings (e.g., 3×3 grid, 2×4 grid as shown in FIG. 1, 1×8 strip) or the template 1 may be patient-specific and the marker pattern may be based on previously performed analysis of the patient's condition. For example, for a patient susceptible to having a seizure, and if the patient's seizure foci are known, the template 1 may be customized to correspond to the patient's estimated foci “shape”.

In preferred embodiments, the markers 4 identify locations for openings to be in created in the patient's skull for the placement of an electrode array. The electrode array, or arrays, is generally in a pattern that corresponds to the marker pattern.

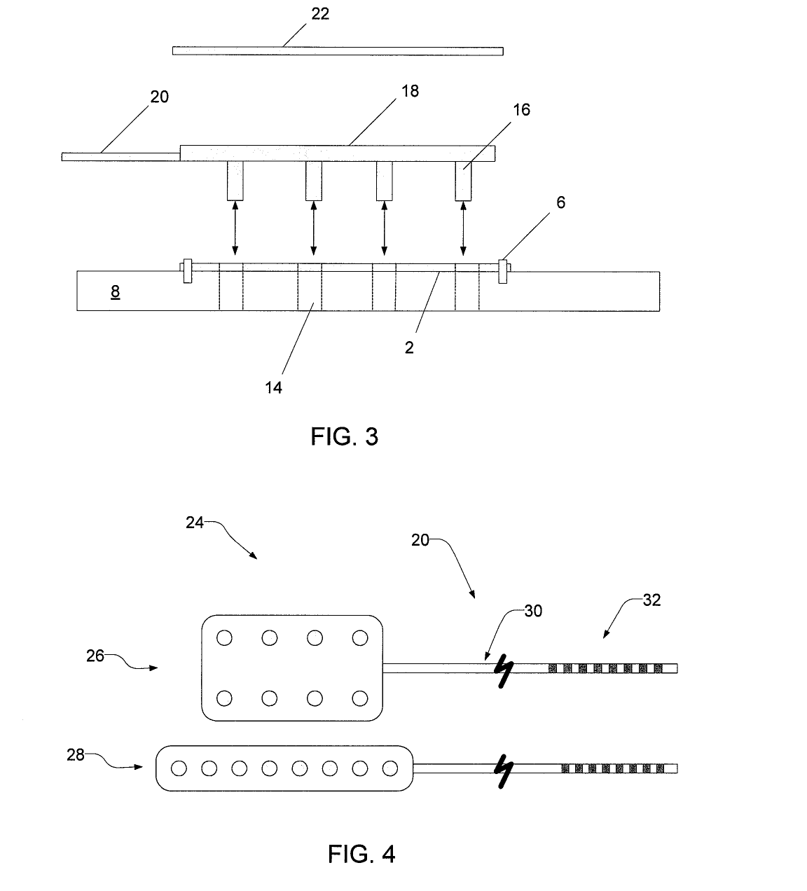

The template 1 may further comprise one or more attachments 6 for removably or permanently coupling the template 1 to an outer surface of the patient's skull 8. The anchors 6 may comprise adhesive, screws, double sided tape, or other biocompatible means to couple the template 1 to the skull 8. FIGS. 1 and 3 illustrate attachments 6 as bone screws, but any conventional type of attachment may be used. The bottom surface of the template 1 may be in direct contact with an exterior surface of the skull 8, as shown in FIG. 3. The substrate may not, however, be in direct contact with a surface of the skull if, for example, an adhesive or double sided tape couples the bottom surface of the template 1 to an exterior surface of the skull 8. In some embodiments, the template 1 may have an adhesive layer with a peel-off protective covering to couple, either permanently or temporarily, the template assembly to the skull (not shown).

The template substrate 2 is generally flexible and substantially flat, but may be curved to correspond to the general curvature of the outer surface of the patient's skull 8, if desired. The template substrate 2 may also have different portions with varying degrees of curvature to suitably mate with the skull 8. If desired, a mold of a portion of the patient's skull may also be taken to better fit the template substrate 2 to the skull 8.

The template substrate 2 may be composed of any number of different materials. The materials should generally be biocompatible, MRI safe, and preferably does not interfere with CT scans or other imaging techniques. Some useful materials include stainless steel, titanium, polymers, or other substantially flexible materials.

The template substrate 2 may include markings or other indications that indicate to the physician which side is to be placed on the outer surface of the skull (e.g., text that says “this side up” or “this side down”). Furthermore, particularly for the patient-specific templates in which the electrode arrays may or may not be the same, the templates may include an indication or instruction for the physician as to which portion of the skull the template should be placed. For example, if the physician is placing two electrode arrays over each of the patient's temporal lobes, the electrode arrays for each of the temporal lobes may indicate whether the template 1 is for a mesial or lateral strip and/or indicate which lobe the template is for.

FIG. 3 shows openings or channels 14 that have been created in the patient's skull 8 with, for example, a drill bit. The template substrate 2 may be left in place on the skull or it may be removed. If the template is left in place, as shown in FIG. 3 and described above, the template 1 may have a mating surface that is configured to contact and help secure the individual electrodes 16, shown coupled to electrode substrate 18, in place.

In some embodiments the template 1 may comprise means for receiving one or more leads 20 of the electrode array, such as, for example, pre-formed grooves or channels sized and shaped to receive the one or more leads from the electrode array. If desired, the leads may further be encapsulated by template lid 22 that can be shaped to mate with the template to further protect the electrodes and any leads that may be connected to the array.

The electrodes 16 that may be used with the template 1 may extend only through a portion of the skull, through one layer of the table, through at least a portion of two layers of the table, may extend into an epidural space, contact CSF, or may even extend through the dura mater (e.g., subdural), and/or into the patient's brain (e.g., deep brain electrodes). The electrode array may be used to monitor brain signals, such as, for example, EEG and MEG, deliver neuromodulation signals to the brain, or a combination thereof.

Some exemplary configurations of electrode arrays 24 are shown in FIG. 4. Each of the illustrated electrode arrays has eight electrode contacts so as to provide 16 channels for monitoring brain signals. The electrode contacts may be bipolar or referential. It should be appreciated however, that while FIG. 4 illustrates 16 channels that are distributed over two electrode arrays, any number of electrode arrays that have any number of contacts may be used with the present invention. In most embodiments, however, the system typically includes between about 1 and about 256 channels, and preferably between about 1 and about 32 channels, and more preferably between 8 and 32 channels that are distributed over 1 array and about 4 arrays. The array pattern and number of contacts on each array may be configured in any desirable pattern. The markers on the template may be in a pattern that corresponds to the electrode pattern.

FIG. 4 specifically illustrates a 2×4 grid electrode array 26 and a 1×8 strip electrode array 28. The electrode arrays may be coupled to an implanted device (described below) with leads 20. The leads of each of the electrode arrays will typically have a common lead body 30 and connector 32. The connector 32 may take any conventional or proprietary form, such as those based on SCS/ICD technology. The electrode arrays could be used in either a bipolar or monopolar configuration.

The electrodes that are implanted into the openings created using the template can also be wireless devices, similar to the Advanced Bionics BION device or the devices described in co-assigned U.S. Patent Application Ser. No. 60/805,710, filed on Jun. 23, 2006. Such wireless electrodes may be in wireless communication with a device implanted in the patient or external to the patient.

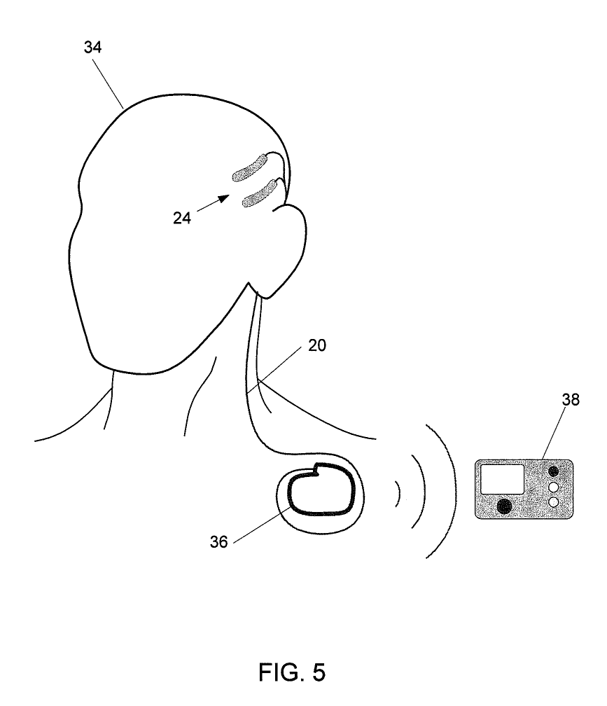

FIG. 5 illustrates one example of a system for which the template of the present invention may be used. The system can be used to monitor a neurological condition of patient 34 for purposes of estimating a subject's susceptibility for a neurological event. The system of the illustrated embodiment provides for substantially continuous sampling and analysis of brain wave electrical signals.

The system comprises one or more electrode arrays 24 that are implanted in openings created in the skull, and the arrays are configured to measure signals from the patient 34. In one embodiment, one or more of the electrode arrays 24 will be implanted adjacent a previously identified epileptic focus, a portion of the brain where such a focus is believed to be located, or adjacent a portion of a seizure network.

The electrodes are shown electrically joined via lead 20 to a communication unit 36, but could be in wireless communication with the communication unit or other external devices. In one embodiment, the lead 20 and the communication unit 36 will be implanted in the subject. For example, the communication unit 36 may be implanted in a sub-clavicular cavity of the subject. In alternative embodiments, the lead 20 and communication unit 36 may be implanted in other portions of the subject's body (e.g., in the head) or attached to the subject externally.

The communication unit 36 may be configured to facilitate the sampling of brain signals from the electrodes. Sampling of brain activity is typically carried out at a rate above about 200 Hz, and preferably between about 200 Hz and about 1000 Hz, and most preferably at or above about 400 Hz. The sampling rates could be higher or lower, depending on the specific features being monitored, the subject, and other factors. Each sample of the subject's brain activity is typically encoded using between about 8 bits per sample and about 32 bits per sample, and preferably about 16 bits per sample. In alternative embodiments, the communication unit 36 may be configured to measure the signals on a non-continuous basis. In such embodiments, signals may be measured periodically or aperiodically.

An external device 38 may be carried external to the body of the patient. The external device 38 can receive and stores signals, including measured brain signals and possibly other physiological signals, from the communication unit 36. Communication between the external device 38 and the communication unit 36 (or the wireless electrodes) may be carried out through wireless communication, such as a radiofrequency link, infrared link, optical link, ultrasonic link, or other conventional or proprietary wireless link. The wireless communication link between the external device 38 and the communication unit 36 may provide a one-way or two-way communication link for transmitting data.

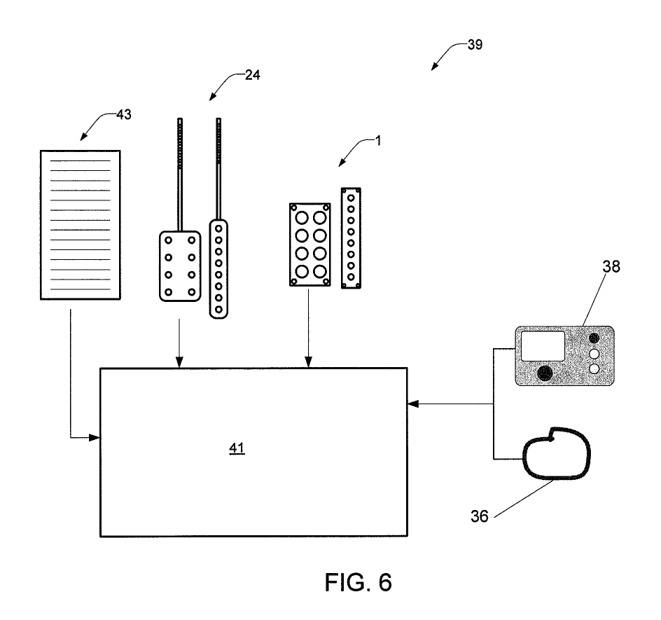

FIG. 6 illustrates an example of a packaged system or kit 39 that is embodied by the present invention. The packaged system 39 may include a package 41 that includes one or more compartments for receiving one or more electrode arrays 24, one or more templates 1 that correspond to the orientation of the electrode contacts of the corresponding electrode arrays, and instructions for use (IFU) 43 that describe any of the methods described herein. If desired, the kit 39 may also include the implantable communication unit 36 and/or the external unit 38. Such components may be in their own packaging (not shown) or in a compartment within the package 41.

The methods of the present invention are directed toward methods of implanting a plurality of electrodes at locations in a patient's skull. In a preferred embodiment, the method is directed toward implanting an electrode array substantially over a seizure foci or seizure network to monitor brain signals that are predictive or indicative of a patient's susceptibility of a seizure (e.g., seizure prediction or seizure detection) and/or to deliver stimulation to the patient's brain. The electrode array may, however, be used for any other type of system or method that is used to monitor brain activity. Furthermore, the present invention is relevant to any type of head-mounted electrodes (e.g., pressure sensor, CSF monitors, temperature monitors, stimulation electrodes, etc.) that embed into or through the skull.

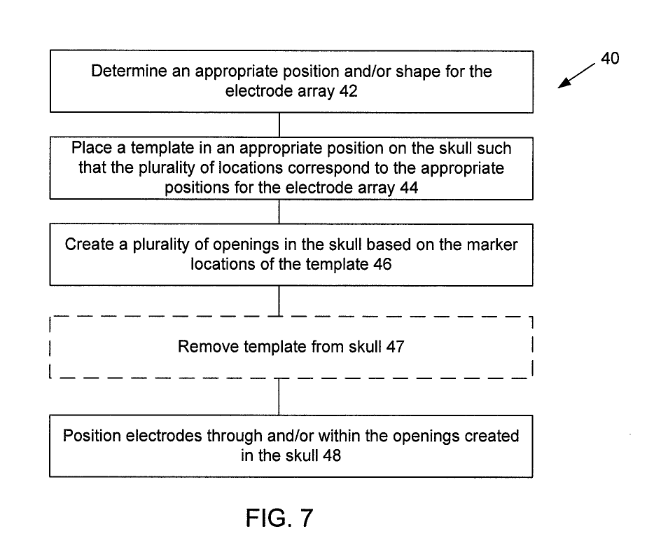

The following steps are directed toward one preferred non-limiting embodiment of a method 40 encompassed by the present invention. Other methods of the present invention may include additional or fewer steps than described. First as shown in FIG. 7, an appropriate position for an array of electrodes will be determined 42. A template, including a pattern of markers identifying a plurality of locations on the skull for creating a plurality of openings, will then be placed in an appropriate position on the skull such that the plurality of locations correspond to the appropriate positions for the plurality of electrodes 44. A plurality of holes or openings in the skull are then created based on the marker locations 46. The markers may be a plurality of openings or channels, and creating the openings in the skull can include drilling holes or openings in the skull through openings in the template. It is also possible that channels or openings in the template may be used to mark the locations on the skull where the openings are to be drilled and then create openings using those markings. When markings are made on the skull using the template, the template may be removed after the markings are made and before creating the hole or openings in the skull (optional) 47. The template may be removed from the skull or it can be left in place. The electrodes are then positioned in the holes or openings created in the skull 48.

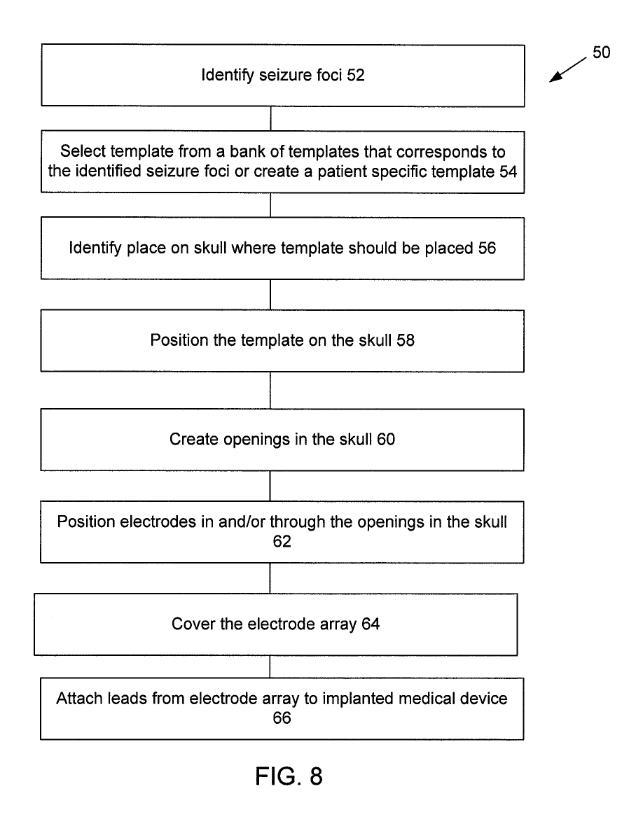

In some embodiments the method 50 is particular for implanting a plurality of electrodes in a patient susceptible to having a seizure. An exemplary method is shown in FIG. 8. First, seizure foci are identified using, for example, intracranial EEG monitoring, scalp EEG monitoring, spike triggered fMRI, and/or SPECT 52. The term “seizure foci” encompasses the groups of neurons that are responsible for the epileptogenic activity. The term also encompasses the neurons or groups of neurons that are providing signals that are predictive of the onset of the subject's seizure activity. Once the seizure foci are identified, an appropriate template is selected from a bank of templates, or a patient-specific template is created that will allow the electrodes to be positioned appropriately above or adjacent the identified seizure foci 54. Markings may then be made on the outer surface of the patient's skull to identify where the template should be positioned 56. The markings may be relative to landmarks on the skull. The template is then coupled, either temporarily or permanently, to an outer surface of the patient's skull so that the markings for the holes or openings in the skull are substantially aligned with the previously identified seizure foci 58. A plurality of holes or openings for the electrodes are then created using an appropriate bone drill, or the template can be used to mark locations on the skull which are then used to create a plurality of holes or openings in the skull 60. The template may be left in place or it may be removed from the surface of the patient's skull before or after creation of the plurality of holes or openings in the skull. Electrodes are then positioned into the holes or openings in the skull 62. As noted above, the holes may extend through the entire table of the skull or only through a portion of the skull table. If desired, a template lid may be placed over the electrode array and template to further cover and protect the electrodes and leads that may be part of the array 64. If needed, the leads may then be tunneled and attached to an implanted medical device 66. In some embodiments, the implanted medical device is positioned in a sub-clavicular pocket. As such, the leads may be tunneled between the skull and the scalp and down through the neck to the sub-clavicular pocket. An implanted device, however, could be coupled to the template and positioned above the skull and below the scalp, such as the Medtronic DBS® and/or the NeuroPace RNS.

The electrodes may be used in other brain activity monitoring/stimulation systems such as sleep apnea and other sleep disorders, migraine headaches, depression, Alzheimer's, Parkinson's Disease, essential tremor, dementia, bipolar spectrum disorders, attention deficit disorder, stroke, cardiac disease, diabetes, cancer, eating disorders, or the like.

A description of some systems that may use the electrodes described herein to delivery a neuromodulation output to a patient are described in commonly owned U.S. Pat. Nos. 6,366,813 and 6,819,956, U.S. Patent Application Publication Nos. 2005/0021103 (published Jan. 27, 2005), 2005/0119703 (published Jun. 2, 2005), 2005/0021104 (published Jan. 27, 2005), 2005/0240242 (published Oct. 27, 2005), 2005/0222626 (published Oct. 6, 2005), and U.S. patent application Ser. Nos. 11/282,317 (filed Nov. 17, 2005), 11/321,897, 11/321,898, and 11/322,150 (all filed Dec. 28, 2005).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. For example, instead of an electrode, it may be desirable to position a catheter or other output from an implanted drug dispenser so as to position the one or more localized drug delivery outputs over the desired portion of the patient's brain. It is intended that the following claims define the scope of the invention and that methods and structures within the scope of these claims and their equivalents be covered thereby.

What is claimed is:

1. A template for creating a plurality of openings at locations in the skull of a patient, wherein the plurality of openings are configured to receive an electrode array, the template comprising:
a substrate configured to be coupled to an outer surface of the patient's skull; and
a plurality of markers in a pattern on the substrate, the markers configured to identify locations for creating a plurality of openings in the skull, the openings being configured to receive an electrode array comprising a plurality of electrodes that are positioned in a pattern that corresponds to the pattern of the plurality of markers.

2. The template of claim 1 wherein the substrate is configured to be removably coupled to the outer surface of the skull.

3. The template of claim 1 wherein the substrate is configured to be permanently coupled to the outer surface of the skull.

4. The template of claim 1 wherein the plurality of markers are configured to identify patient-specific locations.

5. The template of claim 1 wherein the locations are based on a location of a seizure foci.

6. The template of claim 1 wherein the plurality of markers comprise a plurality of channels in the substrate, wherein the plurality of channels are adapted to receive a drill element.

7. The template of claim 1 wherein the plurality of markers comprise a plurality of visual markings on the substrate.

8. The template of claim 1 wherein the plurality of electrodes are configured to monitor brain signals.

9. The template of claim 1 wherein the plurality of electrodes are configured to deliver a neuromodulation output to the patient's brain.

10. A system for implanting a plurality of electrodes at locations within a patient's skull,
the system comprising:
a template comprising a substrate and a plurality of markers in a pattern, wherein the substrate is configured to be coupled to the outer surface of the skull, and wherein the markers are configured to identify locations at which to create a plurality of openings in the skull; and
an electrode array comprising a plurality of electrodes that are in a pattern that corresponds to the pattern of the plurality of markers,
wherein the plurality of openings in the skull are configured to receive the plurality of electrodes.

11. The system of claim 10 wherein the substrate is configured to be removably coupled to the outer surface of the skull.

12. The system of claim 10 wherein the substrate is configured to be permanently coupled to the outer surface of the skull.

13. The system of claim 10 wherein the plurality of markers are configured to identify patient-specific locations.

14. The system of claim 10 wherein the locations are based on a location of a seizure foci.

15. The system of claim 10 wherein the plurality of markers comprise a plurality of channels in the substrate, wherein the plurality of channels are configured to receive a drill element.

16. The system of claim 10 wherein the plurality of markers comprise a plurality of visual markings on the substrate.

17. The system of claim 10 wherein the plurality of electrodes are configured to monitor brain signals.

18. The system of claim 10 wherein the plurality of electrodes are configured to deliver a neuromodulation output to the patient's brain.

19. The system of claim 10 wherein the electrode array further comprises at least one lead, and wherein the template comprises at least one lead channel adapted to receive the at least one lead.

20. The system of claim 10 further comprising a template lid shaped to mate with the template, wherein the electrode array is configured to be disposed between the template lid and the template when the template lid mates with the template.

21. A method for implanting a plurality of electrodes at locations in a patient's skull, the method comprising:
positioning a template comprising a plurality of markers on an exterior surface of the skull, the markers identifying a plurality of locations on the skull for creating a plurality of holes;
creating the plurality of holes in the skull at the plurality of locations; and
positioning a plurality of electrodes in the plurality of holes.

22. The method of claim 21 further comprising determining a plurality of locations by identifying a seizure foci.

23. The method of claim 22 wherein positioning a plurality of electrodes in the plurality of holes comprises positioning a plurality of electrodes substantially above or over the seizure foci.

24. The method of claim 22 further comprising selecting a template from a plurality of templates based on the determination of the plurality of locations.

25. The method of claim 21 wherein positioning a template comprises positioning a patient-specific template comprising patient-specific markers.

26. The method of claim 21 wherein the markers comprise a plurality of channels in the template, and wherein creating a plurality of holes comprises drilling into the skull through the plurality of channels.

27. The method of claim 21 wherein the markers comprises a plurality of channels, the method further comprising
creating a visual reference on the exterior surface of the skull at the identified locations after positioning the template on the exterior surface of the skull, and
removing the template from the exterior surface of the skull before creating a plurality of holes.

28. The method of claim 21 further comprising positioning a lid on the template, thereby securing the electrode array.
